(19) United States
(12) Patent Application Publication
(10) Pub. No.: US 2007/0032448 A1
(43) Pub. Date: Feb. 8, 2007

(54) Sugar modified nucleosides as viral replication inhibitors

(76) Inventors: Zhi Hong, Irvine, CA (US); Hadylin An, Carlsbad, CA (US); Yili Ding, Fountain Valley, CA (US); Jean-Luc Girardet, Aliso Viejo, CA (US); Weidong Zhong, Laguna Niguel, CA (US)

Correspondence Address: THELEN REID BROWN RAYSMAN & STEINER LLP, 900 THIRD AVENUE, NEW YORK, NY, 10022, US

(21) Appl. No.: 10/535,742

(22) PCT Filed: Oct. 2, 2002
(86) PCT No.: PCT/US02/31556

§ 371 Date: Sep. 25, 2006

Related U.S. Application Data
(60) Provisional application No. 60/350,296, filed on Jan. 17, 2002.
(60) Provisional application No. 60/391,800, filed on Jun. 26, 2002.

(51) Int. Cl. A61K 31/7076

(52) U.S. Cl. 514/45; 514/49; 536/26.1; 536/27.3; 536/27.4; 536/28.3

(57) ABSTRACT

Various 2′-modified nucleoside analogs and corresponding prodrugs are provided, and particularly contemplated methods of use include use as antiviral agents, and especially as antiviral agents against HCV.

### FIELD OF THE INVENTION

The field of the invention is viral replication inhibitors, and especially RNA viral replication inhibitors.

### BACKGROUND OF THE INVENTION

Numerous nucleosides are known to interact with various biological targets. Thus, numerous approaches have been undertaken to employ nucleoside analogs as antiviral agents or antimetabolites, and depending on the particular nucleoside analog, the desired mode of action may vary considerably.

For example, many nucleoside analogs can be phosphorylated to monophosphates by nucleoside kinases after the nucleoside analog enters the cell. These monophosphates may then be further phosphorylated by nucleoside monophosphate kinases and nucleoside diphosphate kinases to give nucleoside triphosphates. Once a nucleoside analog is converted to its triphosphate inside the cell, it can be incorporated into DNA or RNA, thereby interrupting gene expression by chain termination or by interfering with the function of the modified nucleic acids.

Moreover, certain nucleoside analog triphosphates are relatively potent, competitive inhibitors of DNA or RNA polymerases, which can significantly reduce the rate at which the natural nucleoside can be incorporated. Among other compounds, many anti-HIV nucleoside analogs fall into this category, including 3′-C-azido-3′-deoxythymidine, 2′,3′-dideoxycytidine, 2′,3′-dideoxyinosine, and 2′,3′-didehydro-2′,3′-dideoxythymidine.

In another example, various purine-type and other nucleoside analogs can also act in other ways, including causing apoptosis of cancer cells and/or modulating immune systems. In addition to nucleoside antimetabolites, a number of nucleoside analogs that show very potent anticancer and antiviral activities act through still other mechanisms. For example, some well-known nucleoside anticancer drugs are thymidylate synthase inhibitors such as 5-fluorouridine, and adenosine deaminase inhibitors such as 2-chloroadenosine. Alternatively, neplanocin A, is an inhibitor of S-adenosylhomocysteine hydrolase, which shows potent anticancer and antiviral activities.

Unfortunately, many nucleoside analogs that can inhibit tumor growth or viral infections are also toxic to normal mammalian cells, primarily because these nucleoside analogs lack adequate selectivity between the normal cells and the virus-infected host cells or cancer cells. For this reason many otherwise promising nucleoside analogs fail to become therapeutics in treatment of various diseases.

Selective inhibition of cancer cells or host cells infected by viruses has been an important subject for some time, and tremendous efforts have been made to search for more selective nucleoside analogs. There are numerous publications, patent applications, and patents that disclose a wide variety of compounds that allegedly act as potent antiviral and/or antineoplastic agents. However, upon closer examination, a significant number of those compounds exhibit undesirably low antiviral and/or antineoplastic activity, if any activity at all.

Thus, although there are numerous nucleoside analogs and methods known in the art, all or almost all of them suffer from various disadvantages. Therefore, there is still a need to provide improved nucleoside analogs and methods for specific and potent antiviral and/or antineoplastic activity.

### DETAILED DESCRIPTION

The inventors have discovered that particular nucleoside analogs exhibit surprisingly significant antiviral activity, while chemically closely related nucleoside analogs do not exhibit any appreciable activity.

In particular, the inventors discovered that compounds according to Formula 1 or Formula 2 have significant antiviral activity, while similar compounds exhibit dramatically reduced, if any antiviral activity: 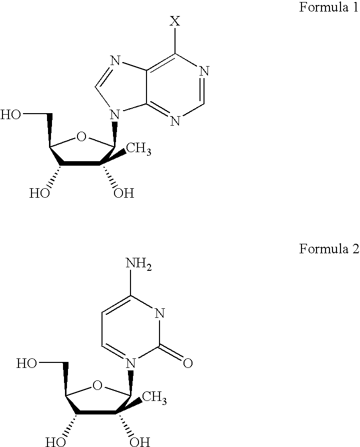
wherein in such compounds X is selected from the group consisting of NH₂, NHCH₃, N(CH₃)₂, OCH₃, and SCH₃.

Contemplated variations of the compounds according to Formulae 1 and 2 with potential antiviral and/or antineoplastic activity especially include modifications on the sugar and/or heterocyclic base portion. For example, where the sugar portion is modified, it is contemplated that a suitable modification is replacement of one or more hydrogen atoms in the 2′-beta methyl group with a halogen, and substitution of the sugar oxygen with a sulfur atom or a methylene group. Further particularly contemplated modifications include (mono-, di-, tri-, and poly-) phosphates and phosphonates coupled to the sugar via the C5′-atom, all of which may or may not be further modified (e.g., replacement of an oxygen with a sulfur, esterified with an additional group, etc.).

In another example, contemplated modifications on the heterocyclic base portion of the compounds according to Formula 1 may include small (i.e., M_{w} less than 150) polar and non-polar groups, which may be coupled to the 6-position of the heterocyclic base via a carbon or heteroatom, including sulfur, oxygen, or selenium. Moreover, it should be recognized that additional substituents may be added to the heterocyclic ring system, and an especially preferred position includes the 8-position (e.g., with a halogen or other small substituent). Similarly, while it is generally preferred that the heterocyclic base of Formula 1 is a purine, deazapurines (and particularly 3-, 7- and 9-deazapurines) and azapurines (particularly 8-azapurines) are also contemplated.

In a further example, contemplated modifications on the heterocyclic base portion of the compounds according to Formula 2 may include small (i.e., M_{w} less than 150) polar and non-polar groups, which may be coupled to the 5-position of the heterocyclic base. Especially contemplated substituents include halogens. Furthermore, while it is generally preferred that the heterocyclic base of Formula 2 is a pyrimidine, deazapyrimidines (particularly 1-deazapyrimidines) and azapyrimidines (particularly 5-, 6-azapyrimidines) are also contemplated. Similarly, it should be recognized that the heterocyclic base of Formula 2 need not be aromatic.

Thus, contemplated compounds may therefore include a group X instead of the NH₂ group at the 4 position of the cytidine of Formula 2 and further include a group R at the 2 position of Formula 1, wherein X may be NH₂, NHCH₃, NH(CH₃)₂, OCH₃, SCH₃, OH, SH, and wherein R may be H, or NH₂ to include various G and U derivatives.

It should be especially noted that some of the compounds according to Formulae 1 and 2 above (and some of the variations described above) have previously been disclosed in WO01/90121 to Novirio as falling within an extremely broadly defined class of nucleoside analogs with alleged antiviral activity. However, as extensive research and numerous data (see below) on such classes of nucleoside analogs demonstrated, particular biological activities of the compounds belonging to those classes is extremely unpredictable in light of even minor structural variations. Consequently, it should be recognized that the inventors in the Novirio application have not recognized, identified, or appreciated particular biological activities of the selected compounds presented herein (i.e., compounds of Formulae 1 and 2, and their variations as described above).

While it is generally contemplated that administration of such compounds may be systemic or specific to a particular organ, it is typically preferred that the above compounds may be administered in the form of a prodrug. Particularly suitable prodrug forms of the above compounds may include a moiety that is covalently coupled to at least one of the C2′-OH, C3′-OH, and C5′-OH, wherein the moiety is preferentially cleaved from the compound in a target cell (e.g., Hepatocyte) or a target organ (e.g., liver). While not limiting to the inventive subject matter, it is preferred that cleavage of the prodrug into the active form of the drug is mediated (at least in part) by a cellular enzyme, particularly receptor, transporter and cytochrome-associated enzyme systems (e.g., CYP-system).

Especially contemplated prodrugs comprise a cyclic phosphate, cyclic phosphonate and/or a cyclic phosphoamidate, which are preferentially cleaved in a hepatocyte to produce the compound according to Formula 1 or 2. There are numerous such prodrugs known in the art, and all of those are considered suitable for use herein. However, especially contemplated prodrug forms are disclosed in WO 01/47935 (Novel Bisamidate Phosphonate Prodrugs), WO 01/18013 (Prodrugs For Liver Specific Drug Delivery), WO 00/52015 (Novel Phosphorus-Containing Prodrugs), and WO 99/45016 (Novel Prodrugs For Phosphorus-Containing Compounds), all of which are incorporated by reference herein. Consequently, especially suitable prodrug forms include those targeting a hepatocyte or the liver.

Still further particularly preferred prodrugs include those described by Renze et al. in Nucleosides Nucleotides Nucleic Acids April-July 2001;20(4-7):931-4, by Balzarini et al. in Mol Pharmacol November 2000;58(5):928-35, or in U.S. Pat. No. 6,312,662 to Erion et al., U.S. Pat. No. 6,271,212 to Chu et al., U.S. Pat. No. 6,207,648 to Chen et al., U.S. Pat. No. 6,166,089 and U.S. Pat. No. 6,077,837 to Kozak, U.S. Pat. No. 5,728,684 to Chen, and published U.S. Application with the number 20020052345 to Erion, all of which are incorporated by reference herein. Alternative contemplated prodrugs include those comprising a phosphate and/or phosphonate non-cyclic ester, and an exemplary collection of suitable prodrugs is described in U.S. Pat. No. 6,339,154 to Shepard et al., U.S. Pat. No. 6,352,991 to Zemlicka et al., and U.S. Pat. No. 6,348,587 to Schinazi et al. Still further particularly contemplated prodrug forms arc described in FASEB J. September 2000;14(12):1784-92, Pharm. Res. Aug. 16, 1999:8 1179-1185, and Antimicrob Agents Chemother March 2000 44:3 477-483, all of which are incorporated by reference herein.

Thus, particularly preferred prodrug forms will comprise a moiety covalently coupled to at least one of the C2′-atom, C3′-atom, and C5′-atom, wherein at least part of the moiety is preferentially cleaved from the compound in a target cell or target organ. As used herein, the term “preferentially cleaved . . . in a target cell or target organ” means that cleavage occurs in a particular target cell or target organ at a rate that is at least 3 times, more typically at least 10 times, and most typically at least 50 times higher than in a non-target cell or non-target organ. The term “target cell” or “target organ” as used herein refers to a cell or organ that is infected with a virus, and especially includes a hepatocyte infected with an HCV virus. Cleavage may be mediated by enzymes (but also by non-enzymatic processes, e.g., via reductive cleavage), and it is particularly preferred that enzymatic cleavage is mediated by a liver-specific enzyme system (e.g., CYP system). Consequently, it should be appreciated that certain prodrug forms of contemplated compounds may be cleaved in a target cell and/or target organ to provide a nucleotide analog.

An exemplary preferred prodrug of contemplated compounds may therefore include a moiety according to Formula M1 or M2 (covalently coupled to the compound, typically to the C5′-atom, C2′-atom, and/or C3′-atom):

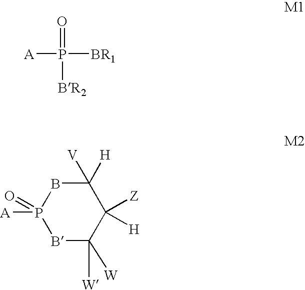

wherein A in M1 or M2 is O or CH₂ and replaces the 5′-OH group of the compound of Formula 1 or Formula 2; B and B′ are independently O or NH, and where B is NH then R₁ or R2 is an amino acid that forms a peptide bond with the N atom of the NH; and V, W, and W′ are independently hydrogen, alkyl, alkenyl, alkynyl, aryl, alkaryl, each of which is optionally substituted, and Z is hydrogen, CHWOH, CHWOCOW′, SW, or CH₂aryl. Especially preferred compounds according to Formula M2 are those in which in A is O or CH₂, B and B′ are independently O or NH, and in which Z, W, and W′ are H and V is m-Chloro-phenyl. Therefore, it should be recognized that especially contemplated compounds may be included in a pharmaceutical composition wherein contemplated compounds are present at a concentration effective to inhibit viral replication, and especially viral replication of the hepatitis C virus. The term “inhibit viral replication” as used herein refers to a reduction in at least one of the initiation of viral nucleic acid synthesis, chain elongation of viral nucleic acid synthesis, processing of viral nucleic acids within a virus infected cell, and viral protein processing/assembly.

Consequently, it should be recognized that a method of treating a viral infection in a mammal will include a step in which at least one of the contemplated compounds is presented to a cell in a concentration effective to reduce viral propagation. The term “viral propagation” as used herein refers to a viral entry into the cell, viral replication, transcription and/or translation of viral genes, integration of viral nucleic acid into the cell genome, viral protein processing, viral protein assembly, and/or viral exit from the host cell.

In particularly preferred methods of treating a viral infection, the viral infection includes an organ inflammation, and preferably a liver inflammation. Consequently, contemplated cells particularly include hepatocytes, and especially contemplated viruses include those belonging to the family of Flaviviridae (e.g., Hepatitis C virus). In still further contemplated aspects, the step of presenting may comprise intracellular presentation as well as extracellular presentation.

Furthermore, it is contemplated that contemplated compounds may be administered as a prodrug to the mammal, wherein the prodrug is converted to the compound in the mammal, and it is particularly preferred that the prodrug is preferentially converted to the compound in the liver (e.g., prodrug comprises ester bonds (e.g., cyclic phosphate, cyclic phosphonate or a cyclic phosphoamidates) that is cleaved to yield the compound).

In yet further contemplated aspects, it should be recognized that contemplated compounds may be administered with a second pharmacological molecule in a manner such that the second pharmacological molecule and the contemplated compound are present in the mammal at the same time. Particularly preferred second pharmacological molecules are selected from the group consisting of ribavirin, interferon-alpha, interferon-gamma, and a molecule that induces expression of an interferon-alpha or interferon-gamma into the mammal.

Synthesis of Contemplated Nucleoside Analogs and closely related Compounds

Compounds 1-6 (see Scheme 1) were prepared based on the reported procedures (Cappellacci, L.; Barboni, G.; Palmieri, M.; Pasqualini, M.; Grifantini, M.; Costa, B.; Martini, C.; Franchetti, P. J. Med. Chem. 2002, 45, 1196-1202).

General procedure for the synthesis of compounds P1-P5. A mixture of compound 6 and liquid ammonia (neat), methylamine, dimethylamine, thiomethanol (NaOH), or methanol (NaOH) in DMF were refluxed for 5 hours under nitrogen atmosphere. The reaction mixture was concentrated and purified by flash chromatography on a silica gel. The resultant compounds were dissolved in methanol and treated with 10% Pd/C in the presence of ammonium formate at elevated temperature. The cooled reaction mixture was concentrated, and the residue was purified by flash chromatography on a silica gel column providing the desired products P1-P5.

Compounds 8 and 10-12 (see Scheme 2) were synthesized by the reported procedures (Wolfe, M. S.; Harry-O'Kuru, R. E. Tetrahedron Lett. 1995, 36, 7611-7614; Harry-O'kuru, R. E.; Smith, J. M.; Wolfe, M. S. J. Org. Chem. 1997, 62, 1754-1759). Compound 16 was synthesized based on the reported procedure (Franchetti, P.; Gappellacci, L.; Marchetti, S.; Trincavelli, L.; Martini, C.; Mazzoni, M. R.; Lucacchini, A.; Grifantini, M. J. Med. Chem. 1998, 41, 1708-1715).

1,2,3,5-Tetra-O-benzoyl-2-C-ethyl-α/β-D-ribofuranose (9). A solution of ethylmagnesium bromide (1 M in THF, 50 mL, 50 mmol) in 100 ml of anhydrous THF was cooled to −78° C. in a dry ice-acetone bath under argon. A solution of 1,3,5-tri-O-benzoyl-2-keto-β-D-ribofuranose (8) (4.6 g, 10 mmol) in 30 ml of anhydrous THF was added dropwise over 30 min. The resultant mixture was stirred at −78° C. for 5 h. The dry ice/acetone bath was removed and 100 mL of saturated NH₄Cl was poured into the reaction mixture. After being allowed to warm to ambient temperature, the organic phase was separated, and the water phase was extracted three times with ethyl acetate. The combined organic phase was dried (Na₂SO₄) and concentrated to provide 4.6 g of a viscous yellow oil. This material was dissolved in 250 mL of dry CH₂Cl₂. To the solution were added 2.50 g (20.5 mmol) of (dimethylamino)pyridine, 4.60 mL (39.6 mmol) of benzoyl chloride, and 25 mL of distilled Et₃N. After being stirred for 7 h at ambient temperature, the reaction mixture was poured into ether and washed with 1 N HCl, saturated NaHCO₃, and brine. The organic phase was dried (Na₂SO₄), filtered, and concentrated to give 5 g of syrup product, which was directly used for the next step without further purification.

1,2,3,5-Tetra-O-benzoyl-2-C-cyclopropyl-α/β-D-ribofuranose (11). To a mixture of magnesium powder (1.7 g, 70 mmol) in 20 ml of anhydrous ether was added dropwise cyclopropyl bromide (5.6 ml, 70 mmol) under argon. After the reaction reached completion, the reaction mixture was cooled to −78° C. in a dry ice-acetone bath under argon, then a solution of 1,3,5-tri-O-benzoyl-2-keto-β-D-ribofuranose (8) (4.6 g, 10 mmol) in 30 ml of anhydrous THF was added dropwise over 30 min. The resultant mixture was stirred at −78° C. for 5 h. The dry ice/acetone bath was removed and 100 mL of saturated NH₄Cl was poured into the reaction mixture. After being allowed to warm to ambient temperature, the organic phase was separated, and the aqueous phase was extracted three times with ethyl acetate. The combined organic phase was dried (Na₂SO₄) and concentrated to provide 5 g of a viscous yellow oil. This material was dissolved in 250 mL of dry CH₂Cl₂. To this solution were added 2.50 g (20.5 mmol) of (dimethylamino)pyridine, 4.60 mL (39.6 mmol) of benzoyl chloride, and 25 mL of distilled Et₃N. After being stirred for 7 h at ambient temperature, the reaction mixture was poured into ether and washed with 1 N HCl, saturated NaHCO₃, and brine. The organic phase was dried (Na₂SO₄), filtered, and concentrated to give 5.5 g of syrup product, which was directly used for the next step without further purification.

9-(1,3,5-Tri-O-benzoyl-2-C-ethyl-β-D-ribofuranosyl)-6-chloropurine (13) and 9-(1,3,5-Tri-O-benzoyl-2-C-ethyl-α-D-ribofuranosyl)-6-chloropurine (13α). To an ice-cooling mixture of crude 1,2,3,5-tetra-O-benzoyl-2-C-ethyl-α/β-D-ribofuranose (9) (5.3 g, 9.3 mmol), 6-chloropurine (2.1 g, 13.9 mmol), and DBU (4.2 ml, 27.7 mmol) in 50 ml of anhydrous acetonitrile was added slowly TMS triflate (6.7 ml, 37 mmol). After stirring at 60° C. for 5 h, the reaction mixture was shaken between saturated NaHCO₃ and CHCl₃. The organic phase was washed with water and dried over Na₂SO₄. After removal of the solvent, the residue was chromatographed (hexane/ethyl acetate, 3:1) to give pure 13 and 13α (85%) in a 4:1 ratio.

9-(1,3,5-Tri-O-benzoyl-2-C-vinyl-β-D-ribofuranosyl)-6-chloropurine (14) and 9-(1,3,5-Tri-O-benzoyl-2-C-vinyl-α-D-ribofuranosyl)-6-chloropurine (14α). 9-(1,3,5-Tri-O-benzoyl-2-C-cyclopropyl-β-D-ribofuranosyl)-6-chloropurine (15). These compounds were synthesized by the same procedure as described above for compounds 13 and 13α.

Representative Procedure for the Synthesis of 2′-C-Ethyladenosine (P6) and 9-(2-C-ethyl-β-D-ribofuranosyl)-6-methoxy-purine (P10), 2′-C-Vinyladenosine (P11), 9-(2-C-vinyl-β-D-ribofuranosyl)-6-methoxy-purine (P15), 2′-C-Cyclopropyladenosine (P16), and 9-(2-C-cyclopropyl-β-D-ribofuranosyl)-6-methoxypurine (P20). A solution of 13 (100 mg) in 5 ml of methanolic ammonia solution was stirred at 60° C. overnight. The solvent was removed, and the residue was chromatographed (chloroform/methanol, 15:1) to give 20 mg of P6 as a white solid and 20 mg of P10.

Typical Procedure for the Synthesis of N⁶-Methyl-2′-C-ethyladenosine (P7), N⁶-Methyl-2′-C-vinyladenosine (P12), and N⁶-Methyl-2′-C-cyclopropylyladenosine (P17). A solution of 13 (100 mg) in 5 ml of 1M methylamine in methanol solution was stirred at room temperature overnight. The solvent was removed, and the residue was chromatographed (chloroform/methanol, 15:1) to give 45 mg of pure compound P7 as white solid.

Typical Procedure for the Synthesis of N⁶-Dimethyl-2′-C-ethyladenosine (P8), N⁶-Dimethyl-2′-C-vinyladenosine (P13), and N⁶-Dimethyl-2′-C-cyclopropylyladenosine (P18). A solution of 13 (100 mg) in 5 ml of 1M methylamine in THF solution was stirred at room temperature overnight. The solvent was removed in vacuo, and the residue was dissolved in 5 ml of methanol. The resultant solution was treated with 10 mg of NaCN. The mixture was stirred at room temperature overnight. The solvent was removed, and the residue was chromatographed (chloroform/methanol, 15:1) to give 45 mg of pure compound P8 as white solid.

Typical Procedrue for the Synthesis of 9-(2-C-Ethyl-β-D-ribofuranosyl)-6-methylmercaptopurine (P9), 9-(2-C-Vinyl-β-D-ribofuranosyl)-6-methylmercaptopurine (P14), and 9-(2-C-Cyclopropyl-β-D-ribofuranosyl)-6-methylmercaptopurine (P19). To a solution of 13 (100 mg) in 5 ml of isopropanol was added 45 mg of sodium thiomethoxide. The resulting mixture was stirred at room temperature overnight. The solvent was removed, and the residue was chromatographed (chloroform/methanol, 15:1) to give 40 mg of pure compound P9 as white solid.

9H-2′-C-Methyl-β-D-ribofuranosyl)adenine (P21) (Scheme 2). 6-Chloro-9H-(2′-C-methyl-2,3,5-tri-O-benzoyl-β-D-ribofuranosyl)purine 16 (40 mg, 0.06 mmol) was treated with methanolic ammonia (15 mL, saturated at 0° C.) and stirred at room temperature for 24 hours in a pressure bottle. The solvent was evaporated to dryness, and the solid residue was purified by silica gel column (CH₂Cl₂-MeOH, 10:1) to yield P21 as a white solid (8 mg, 44%): ¹H NMR (CD₃OD): δ 8.55 (s, 1H), 8.19 (s, 1H), 6.09 (s, 1H), 4.22 (d, 1H, J=9.0 Hz), 4.04 (m, 2H), 3.87 (dd, 1H, J=12.6, 3.0 Hz), 0.89 (s, 3H); ¹³C NMR (CD₃OD): δ 92.0, 83.3, 79.2, 72.9, 59.9.

N⁶-Methyl-9H-(2′-C-methyl-β-D-ribofuranosyl)adenine (P22) (Scheme 2). A solution of 6-chloro-9H-(2′-C-methyl-2,3,5-tri-O-benzoyl-β-D-ribofuranosyl)purine 16 (60 mg, 0.09 mmol) in ethanol (15 mL) was treated with methylamine (1N in THF, 2 mL). This mixture was stirred 75° C. for 12 hours. The solvent was evaporated, and the residue was treated with methanolic ammonia (15 mL, saturated at 0° C.) in a pressure bottle for 24 hours. The solvent was evaporated to dryness, and the solid residue was purified by silica gel column (CH₂Cl₂-MeOH, 10:1) to yield desired product P22 as a yellow foam. ¹H NMR (CD₃OD): δ 8.45 (s, 1H), 8.16 (s, 1H), 6.09 (s, 1H), 4.21 (d, 1H, J=9.0 Hz), 4.04 (m, 2H), 3.87 (dd, 1H, J=12.6, 3.0 Hz), 3.44 (s, 3H), 0.89 (s, 3H); ¹³C NMR (CD₃OD): δ 92.0, 83.3, 79.2, 72.1, 60.0, 38.0, 19.5.

N⁶-Dimethyl-9H-(2′-C-methyl-β-D-ribofuranosyl)adenine (P23) (Scheme 2). A solution of 16 (60 mg, 0.09 mmol) in ethanol (15 mL) was treated with dimethylamine (1N in THF, 2 mL). This mixture was stirred 75° C. for 12 hours. The solvent was evaporated, and the residue was treated with methanolic ammonia (15 mL, saturated at 0° C.) in a pressure bottle for 24 hours. The solvent was evaporated to dryness, and the solid residue was purified by silica gel column (CH₂Cl₂-MeOH, 10:1) to yield the desired product P23 as a yellow oil (25 mg, 83%): ¹H NMR (CD₃OD): δ 8.45 (s, 1H), 8.16 (s, 1H), 6.09 (s, 1H), 4.21 (d, 1H, J=9.0 Hz), 4.04 (m, 2H), 3.87 (dd, 1H, J=12.6, 3.0 Hz), 3.44 (s, 6H), 0.89 (s, 3H); ¹³C NMR (CD₃OD): δ 92.0, 83.3, 79.2, 72.1, 60.0, 38.0, 19.5.

6Thiomethyl-9H-(2′-C-methyl-β-D-ribofuranosyl)adenine (P24) (Scheme 2). A solution of 16 (120 mg, 0.18 mmol) in dry methanol (20 mL) was treated with sodium thiomethoxide (25 mg). This mixture was stirred at 65° C. for 36 hours. The solvent was evaporated to dryness, and the solid residue was purified by silica gel column (CH₂Cl₂-MeOH, 10:1) to yield the desired product P24 as a white solid (40 mg, 66%): ¹H NMR (CD₃OD): δ 8.83 (s, 1H), 8.66 (s, 1H), 6.18 (s, 1H), 4.24 (d, 1H, J=9.0 Hz), 4.07 (m, 2H), 3.88 (dd, 1H, J=12.6, 3.0 Hz), 2.68 (s, 3H), 0.89 (s, 3H); ¹³C NMR (CD₃OD): δ 91.9, 83.3, 79.2, 72.1, 59.8 19.0, 10.7.

6Methoxy-9H-(2′-C-methyl-β-D-ribofuranosyl)adenine (P25) (Scheme 2). A solution of 16 (120 mg, 0.18 mmol) in dry methanol (10 mL) was treated with sodium methoxide (25 mg). This mixture was stirred at 65° C. for 36 hours. The solvent was evaporated to dryness, and the solid residue was purified by silica gel column (CH₂Cl₂-MeOH, 10:1) to yield the desired product P25 as a white solid (25 mg, 83%): ¹H NMR (CD₃OD): δ 8.78 (s, 1H), 8.50 (s, 1H), 6.18 (s, 1H), 4.23 (d, 1H, J=9.0 Hz), 4.06 (m, 2H), 3.88 (dd, 1H, J=12.6, 3.0 Hz), 4.18 (s, 3H), 0.91 (s, 3H); ¹³C NMR (CD₃OD): δ 92.1, 83.3, 79.2, 72.1, 59.8, 53.8, 19.1.

3′-β-Ethynyl-sugar 17 was synthesized based on literature procedure [Bioorg. Med. Chem. Lett. 6, 1887-1892). 3′-β-Methyl-sugar 17 was synthesized based on our reported procedure [J. Med. Chem. 2000, 43, 3704-3713].

Compound 18. To a solution of 17 (2.0 g, 3.78 mmol), 6-chloropurine (1.3 g, 4.158 mmol), and DBU (1.7 mL, 0.657 mmol) in MeCN (10 mL) was added Me₃SiOTf (2.7 ml, 0.015 mmol) slowly with ice cooling. After stirring at 60° C. for 4 h, the reaction mixture was shaken between NaHCO₃ (1M) and CH₂Cl₂, and the organic phase was dried with Na₂SO₄, and evaporated. The crude product was chromatographed (EtOAc) to yield 18 (1.6 g, 2.57 mol, 70%).

Compound 19. To a solution of 17 (100 mg, 0.219 mmol), 6-chloropurine (37 mg, 0.241 mmol), and DBU (100 mg, 0.657 mmol) in MeCN (0.5 mL) was added Me₃SiOTf (0.194 g, 0.876 mmol) slowly with ice cooling. After stirring at 60° C. for 4 h, the reaction mixture was shaken between NaHCO₃ (1M) and CH₂Cl₂, and the organic phase was dried with Na₂SO₄, and evaporated. The crude product was chromatographed (EtOAc) to yield 51 mg of 19.

Compound P26. To a solution of 18 (200 mg, 0.32 mmol) in THF (10 mL) and 1,4-Dioxane (1 mL) was added 2 M solution of methylamine in THF (0.32 mL, 6.4 mmol). The reaction mixture was stirred at 75° C. for 2 h. The solvents were removed under reduced pressure. The mixture was dissolved in methanol (1 mL) and treated with 2 M MeONa in methanol (0.1 mL). After 10 minutes, the reaction mixture was neutralized with Dowex H⁺ resin, and filtered off. The solvent was removed on high vacuum, and the residue was purified on a silica gel column (CH₂Cl₂/MeOH, 9:1) affording P26 (97.6 mg, 90%).

Compound P27. To a solution of 18 (100 mg, 0.160 mmol) in THF (10 mL) was added 2 M solution of dimethylamine in THF (0.16 mL, 3.2 mmol), and the reaction mixture was stirred at 60° C. in a sealed glass bomb for 5 h. The solvents were removed under reduced pressure. The mixture was dissolved in methanol (1 mL) and treated with 2 M MeONa in Methanol (0.15 mL). After 10 minutes, the reaction mixture was neutralized with Dowex H⁺ resin, and filtered off. The solvent was removed on high vacuum, and the residue was purified on a silica gel column (CH₂Cl₂/MeOH, 9:1) affording P27 (13 mg, 53%). Compound P28. To a solution of 18 (50 mg, 0.080 mmol) in DMF (5 ml) was added sodium thiomethoxide (8.4 mg, 0.12 mmol), and the reaction mixture was stirred at room temperature for 24 hours. The solvents were removed under reduced pressure. The mixture was dissolved in methanol (1 mL) and treated with 2 M MeONa in methanol (0.15 mL). After 10 minutes, the reaction mixture was neutralized with Dowex H⁺ resin, and filtered off. The solvent was removed on high vacuum, and the residue was purified on a silica gel column (CH₂Cl_{2/}MeOH, 9:1) affording P28 (13mg, 51%).

Compound P29. To a solution of 19 (30 mg, 0.0574 mmol) in THF (10 mL) was added 2 M solution of methyl amine in THF (0.6 mL, 1.149 mmol), and the reaction mixture was stirred at 75° C. for 2 hours. The solvents were removed under reduced pressure. The mixture was dissolved in methanol (1 mL) and treated with 2 M MeONa in methanol (0.1 mL). After 10 minutes, the reaction mixture was neutralized with Dowex H⁺ resin, and filtered off. The solvent was removed on high vacuum, and the reaction mixture was purified on a silica gel column (CH₂Cl₂/MeOH, 9:1) affording P29 (9 mg, 48%).

Compound P30. To a solution of 19 (25 mg, 0.045 mmol) in THF (10 mL) was added 2 M solution of dimethylamine in THF (0.45, 0.9 mmol), and the reaction mixture was stirred at 100° C. in a sealed glass bomb for 5 hours. The solvents were removed under reduced pressure. The residue was dissolved in methanol (1 mL) and treated with 2 M MeONa in methanol (0.1 mL). After 10 minutes, the reaction mixture was neutralized with Dowex H⁺ resin, and filtered off. The solvent was removed on high vacuum, and the residue was purified on a silica gel column (CH₂Cl₂/MeOH, 9:1) affording P30 (6.9 mg, 50%).

Compound P31. To a solution of 19 (40 mg, 0.0727 mmol) in DMF (5 mL) was added sodium thiomethoxide (30 mg, 0.145 mmol), and the reaction mixture was stirred at room temperature for 24 hours. The solvents were removed under reduced pressure. The mixture was dissolved in methanol (1 mL) and treated with 2M MeONa in methanol (0.1 mL). After 10 minutes, the reaction mixture was neutralized with Dowex H⁺ resin, and filtered off. The solvent was removed on high vacuum, and residue was purified on a silica gel column (CH₂Cl₂/MeOH, 9:1) affording P31 (11 mg, 51%).5′-O-DMT-2′-TBDMS-Inosine (21). To a suspension of inosine (20) (5 g, 18.64 mmol) in DMF (20 ml) and pyridine (50 ml) was added 4,4′-dimethoxytrityl chloride (6.32 g, 18.65 mmol) and DMAP (100 mg). The reaction mixture was stirred at room temperature for 16 h and then concentrated. The syrup was partitioned between EtOAc and water. The organic phase was washed with water, dried, and purified on a silica gel column (EtOAc/MeOH, 1:0→9:1) to give a white solid (7.16 g, 67%).

To a solution of the resulted DMT-derivative (11.38 g, 19.97 mmol) in THF (200 ml) and pyridine (28 ml) was added silver nitrate (8.13 g, 47.86 mmol). After the silver nitrate was dissolved, TBDMS chloride (6.34 g, 42.06 mmol) was added and the mixture was stirred at room temperature for 16 h. The reaction mixture was filtered through a Celite pad and washed with dichloromethane (DCM). The filtrate was evaporated, and the syrup was partitioned between NaHCO₃/H₂O and DCM. The aqueous phase was extracted with DCM. The organic phase was washed with sodium bicarbonate solution, dried, evaporated, and purified on a silica gel column (EtOAc/MeOH, 1:0→9:1). The mixture of 5′-O-DMT-2′-O-TBDMS and 5′-O-DMT-2′-O-TBDMS isomers was concentrated. The solid form was filtered and washed with EtOAc. The filtrate was concentrated and filtered. This was repeated several times until no precipitate formed. The filtrate was concentrated and dissolved in MeOH and treated with triethylamine. The mixture was stirred at 60° C. overnight and evaporated. The syrup was dissolved in warm EtOAc. The mixture was cooled down, and the resulted solid product 21 was filtrated (total yield 7.88 g, 56%).

Compound 22. To an ice-cooled suspension of CrO₃ (3.07 g, 30.76 mmol) in DCM (49 ml) were added Ac₂ (2.91 ml, 30.76 mmol) and Py (4.96 ml, 61.42 mmol). The reaction mixture was stirred at room temperature until homogeneous (15 min) followed by the addition of compound 21 (7 g, 10.22 mmol). The mixture was stirred at RT for 1 h and poured into 1.3 L of cold ethyl acetate. The mixture was stirred at room temperature for 15 min and filtered through a Celite pad. The filtrate was concentrated and purified on silica gel column (hexane/EtOAc 1:4→0:1) to give a foam (5.49, 78.7%).

To a solution of the resultant compound (5.36 g, 7.85 mmol) in THF (65 ml) was added a solution of 3.0 M MeMgI (26.1 ml, 78.5 mmol). The reaction mixture was stirred at room temperature for 1 h. Water was added slowly followed by addition of Celite. The mixture was stirred at room temperature for 5 min and filtered. The filtrate was evaporated, and the solid was partitioned between EtOAc and water. The EtOAc solution was washed with brine, and the aqueous phase was extracted with EtOAc. The organic layer was concentrated, and the residue was purified on a silica gel column (hexane/EtOAc,1:1→0:1) to give product 22 as a colorless foam (2.82 g, 51.4%).

Compound 23. To a solution of Compound 22 (2.8 g, 4.0 mmol) in THF (60 ml) was added a solution of 1.0 M TBAF (4.8 ml, 1.2 mmol). The reaction mixture was stirred at room temperature for 1 h, and Dowex 50 W×12 H⁺ was added (PH z 6). The suspension was stirred for 10 min and filtered. The filtrate was treated with 10% TFA/DCM (24 ml) and stirred at room temperature for 10 min. The mixture was evaporated, co-evaporated with toluene, and neutralized with Dowex 50 W OH⁻ (PH≈7). The suspension was filtered, and the filtrate was evaporated. The resulting syrup was partitioned between water and EtOAc. The organic phase was concentrated to give a residue. A solution of the resulted residue in Py (40 ml) was treated with DMAP (5 mg) and Ac₂O (10 ml). The reaction mixture was refluxed overnight, quenched with MeOH, concentrated and co-evaporated with toluene. The residue was absorbed on silica gel and purified on a silica gel column (hexane/EtOAc 4:1→EtOAc→EtOAc/MeOH 9:1) to give product 23 as a yellow solid (0.5 g, 3 steps 31%).

Compound 24. To a solution of Compound 23 (0.41 g, 1.0 mmol) in acetonitrile (10 ml) was added benzyltriethylammonium chloride (444 g, 2.0 mmol), N,N-dimethylaniline (0.122 ml), and POCl₃ (1.038 ml, 11.0 mmol). The reaction mixture was stirred at 100° C. for 1 h, concentrated, and co-evaporated with toluene. The residue was dissolved in DCM and stirred with ice for 15 min. The organic phase was separated, and the aqueous phase was extracted with DCM. The combined DCM solution was washed with ice-water, dried, and concentrated. The residue was purified on silica gel column (hexane/EtOAc 1:4) to give product 24 as a yellow oil (0.34 g, 80%).

Compound P32. A solution of Compound 24 (0.74 g, 1.734 mmol) in 2.0M methylamine solution in THF (2 ml) was stirred at RT for 2 h followed by the addition of 25% (w/w) sodium methoxide solution in methanol (0.1 ml). The reaction mixture was stirred at room temperature for 1 h, neutralized with HOAc to PH 6-7, and treated with triethylamine to bring the PH to 8. The mixture was concentrated, and the residue was purified on a silica gel column (EtOAc/MeOH 9:1) to give product P32 as a white powder (0.33 g, 65%).

Compound P33. To a solution of Compound 24 (0.1 g, 0.234 mmol) in 1,4-dioxane (0.5 ml) was added 2.0 M dimethylamine solution in THF (2 ml). The reaction mixture was stirred at room temperature for 24 h and concentrated. The residue was dissolved in MeOH (2 ml) and treated with 25% (w/w) NaOMe in MeOH (0.05 ml). The mixture was stirred at room temperature for 2 h and concentrated. The residue was purified on a silica gel column (EtOAc/MeOH, 1:0→9:1) to give an oil which was recrystallized from acetone to give product P33 as a crystalline solid (60 mg, 83%).

Compound P34. To a solution of Compound 24 (0.106 g, 0.248 mmol) in DMF (2 ml) was added NaOSMe (87 mg, 1.24 mmol). The reaction mixture was stirred at room temperature for 24 h and concentrated. The residue was dissolved in MeOH (2 ml) and treated with 25% (w/w) NaOMe in MeOH (0.051 ml). The mixture was stirred at room temperature for 2 h and concentrated. The residue was purified on a silica gel column (EtOAc/MeOH, 1:0→95:5) to give an oil which was dissolved in MeOH and co-evaporated with toluene to give product P34 as a powder (57 mg, 74%).

1-O-Acetyl-2,3,5-tri-O-benzoyl-4-C-ethyl-β-D-ribofuranose (26). 2,3,5-Tri-O-benzoyl-1-O-metyl-4-C-ethyl-β-D-ribofuranose (25) (Esmir Gunic, Jean-Luc Girardet, Zbigniew Pietrzkowski, Cathey Esler and Guangyi Wang, Bioorg. Med. Chem. 2001, 9,163-170) 3.0 g, 5.9 mmol was dissolved in a mixture of acetic acid (14 mL) and acetic anhydride (1.5 mL). Under cooling with ice, sulfuric acid (96%, 165 uL) in acetic acid (1 mL) was added, and the resulting mixture was stirred at room temperature overnight. Ethyl acetate and brine were added, and the organic layer was washed with a saturated aqueous solution of NaHCO₃. The organic extract was dried over sodium sulfate, filtered, and evaporated to dryness. The residue was purified by silica gel chromatography (ethyl acetate (0-3%) in dichloromethane) to give 2.9 g of 26 as a colorless syrup.

6-Chloro-9-(2,3,5-tri-O-benzoyl-4-C-ethyl-β-D-ribofuranosyl)purine (27). DBU (84 uL, 0.56 mmol) was added to a stirred solution of 6-chloropurine (5) (32 mg, 0.21 mmol) and 26 (100 mg, 0.19 mmol) in acetonitrile (2 mL) at 0° C. TMSOTf(135 uL, 0.75 mmol) was added and the reaction mixture was heated at 60° C. for 3 hours. The reaction mixture was cooled to room temperature, and ethyl acetate and brine were added. The layers were separated, and the organic layer was washed with a saturated aqueous solution of NaHCO₃. The organic extract was dried over sodium sulfate, filtered, and concentrated to dryness. The residue was purified by silica gel chromatography to give 120 mg of 27.

6-Amino-9-(2,3,5-tri-O-benzoyl-4-C-ethyl-β-D-ribofuranosyl)purine (P35). Compound 27 (55 mg, 0.09 mmol) was dissolved in methanolic ammonia (2 mL). The reaction mixture was sealed and heated at 80° C. for 48 hours. The mixture was cooled to room temperature, and the solvent was evaporated. The crude material was purified by silica gel chromatography to give P35 (15 mg) as a foam.

6-N,N-Dimethylamino-9-(2,3,5-tri-O-benzoyl-4-C-ethyl-β-D-ribofuranosyl)purine (P36). Dimethylamine (2 M in THF, 300 uL, 0.6 mmol) was added to a solution of 27 (65 mg, 0.10 mmol) in THF (2.4 mL). The reaction mixture was sealed and heated at 70° C. for 48 hours. The mixture was cooled to room temperature, and the solvent was evaporated. The crude material was dissolved in methanolic ammonia (2.5 mL), and the reaction mixture was sealed and stirred at room temperature for 17 hours. The solvent was removed and P36 was recrystallized from methanol (20 mg).

2′-β-C-Methylcytidine P37 (Scheme 6) was synthesized by reported procedure (Tang, X.-Q.; Liao, X.-M.; Piccirilli, J. A. J. Org. Chem. 1999, 64, 747-754; and Herry-O'kuru, R. E.; Smith, J. M.; Wolfe, M. S. J. Org. Chem. 1997, 62, 1754-1759). Compound 38 was synthesized by a similar procedure from 29. Compounds 32 and P39 were synthesized based on the literature procedures (Wolfe, M. S.; Harry-O'Kuru, R. E. Tetrahedron Lett. 1995, 36, 7611-7614, and Herry-O'kuru, R. E.; Smith, J. M.; Wolfe, M. S. J. Org. Chem. 1997, 62, 1754-1759). Compound 33 was synthesized by a similar procedure. Compounds 39 and 42 were obtained by the deprotection of compounds 32 and 33 by ammonia.

Activated Compounds 34 and 35. To a solution of compounds 32 or 33 in DMF in the presence of triethylamine was added 1.2 equivalent of 1,3,5-tris(isopropyl)benzene sulfonyl chloride. The reaction mixture was stirred at room temperature for 5 hours and treated with 1 mL of methanol. The mixture was concentrated and the residue was dissolved in methylene chloride. The layers were separated, and the aqueous phase was extracted with dichloromethane. The organic phase was dried and concentrated. The residue was purified by flash chromatography on a silica gel column to provide the desired products 34 and 35 as white foams, which were kept under nitrogen atmosphere.

General Procedure for the Synthesis of Compounds P40, P41, P43 and P44. A mixture of compounds 34 or 35 and methylamine or dimethylamine in methanol was stirred at 60° C. for 24 hours. The reaction mixture was concentrated, and the residue was purified by flash chromatography on a silica gel column to provide the desired products. 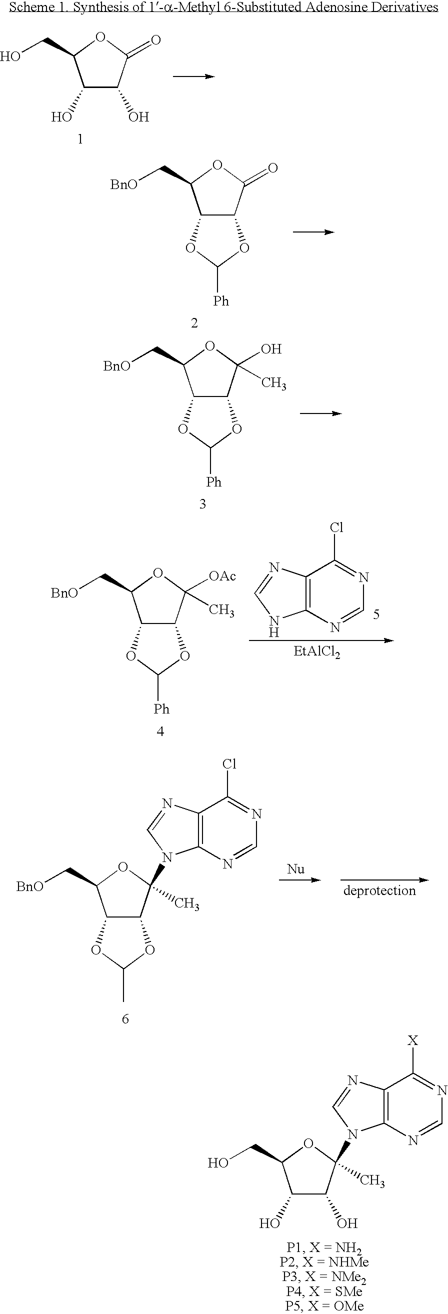 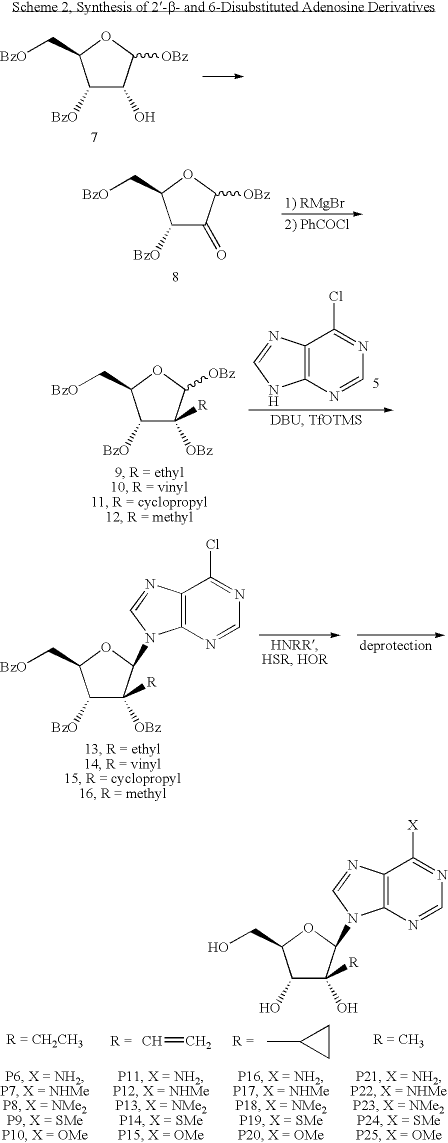 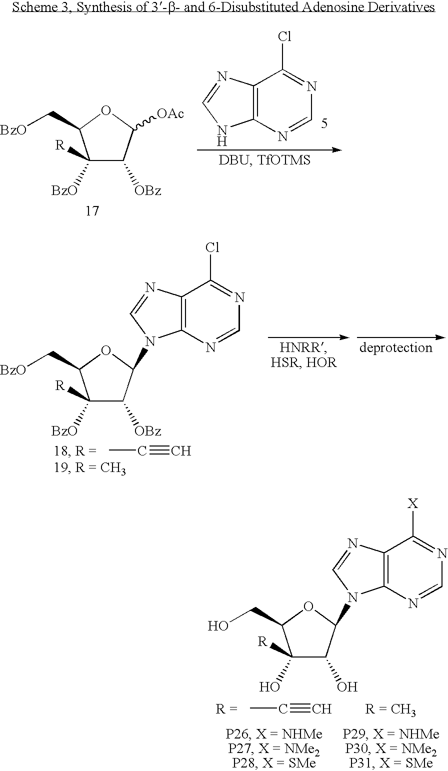 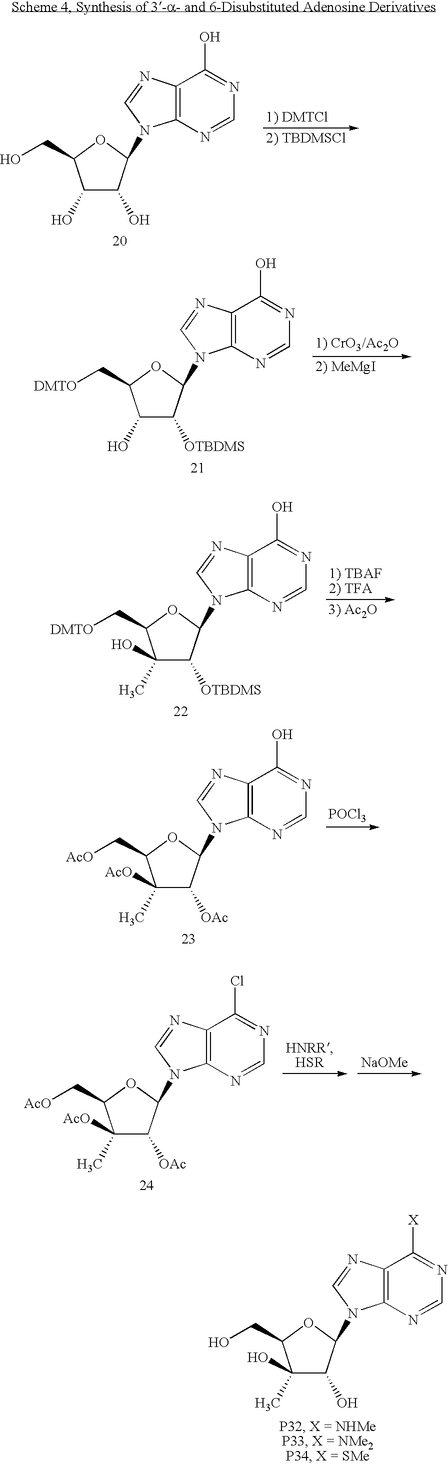 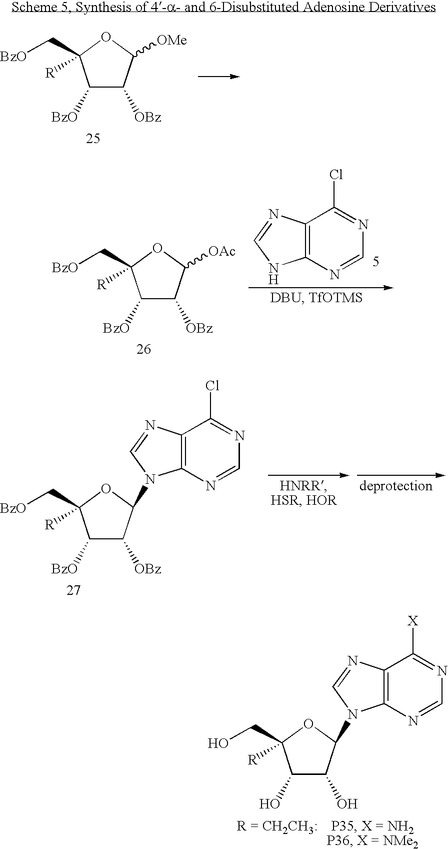 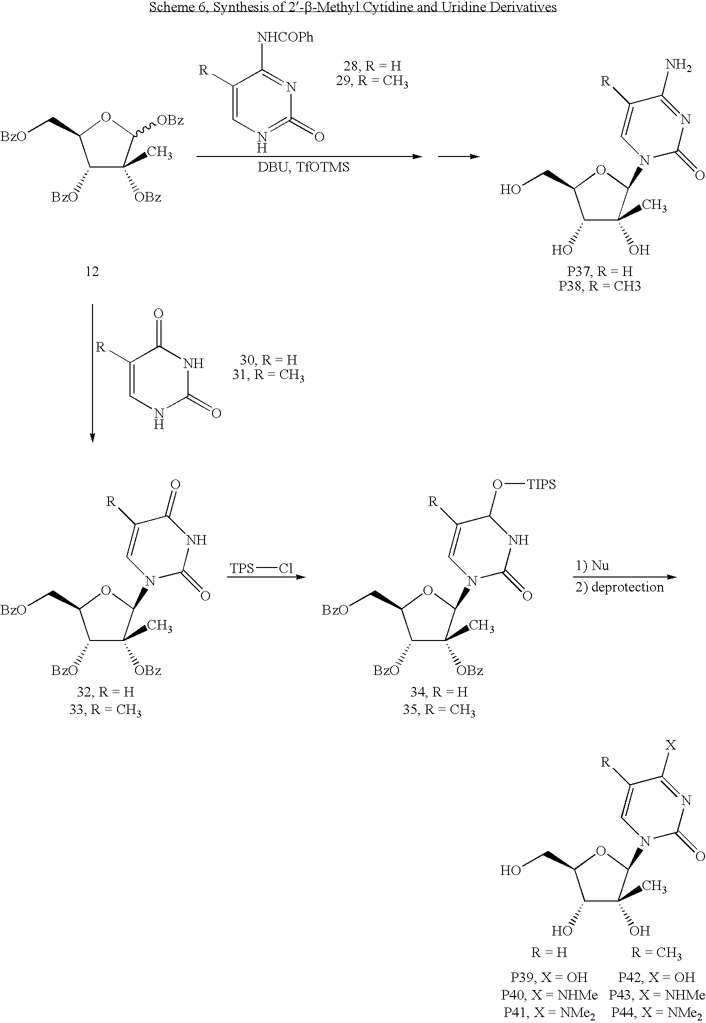
Results

The following results are data obtained using the HCV replicon assay as described below and the letters A, B, and C indicate EC₅₀ values of less than 10 ρM, between 10 and 100 μM, and over 100 μM, respectively. Consequently, it should be especially appreciated that selected compounds, despite their close chemical similarity will exhibit surprisingly different results as RNA polymerase inhibitors.

HCV Replicon Activity of 1′-alpha-6-Substituted Adenosine

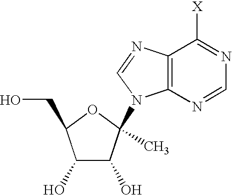

| Compound | X | HCV replicon EC₅₀ |
|---|---|---|
| P1 | NH₂ | To be determined |
| P2 | NHMe | To be determined |
| P3 | N(CH₃)₂ | To be determined |
| P4 | SCH₃ | To be determined |
| P5 | OCH₃ | To be determined |

HCV Replicon Activity of 2′-beta-6-Substituted Adenosine Derivatives

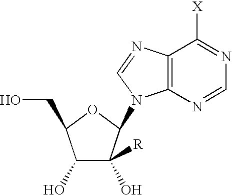

| Compound | 2′-β-R | 6-X | HCV Replicon EC₅₀ |
|---|---|---|---|
| P6 | Isopropyl | NH₂ | C |
| P7 | Isopropyl | NHMe | C |
| P8 | Isopropyl | NMe₂ | C |
| P9 | Isopropyl | SMe | C |
| P10 | Isopropyl | OMe | C |
| P11 | —CH═CH₂ | NH₂ | C |
| P12 | —CH═CH₂ | NHMe | C |
| P13 | —CH═CH₂ | NMe₂ | C |
| P14 | —CH═CH₂ | SMe | C |
| P15 | —CH═CH₂ | OMe | C |
| P16 | CH₂CH₃ | NH₂ | C |
| P17 | CH₂CH₃ | NHMe | C |
| P18 | CH₂CH₃ | NMe₂ | C |
| P19 | CH₂CH₃ | SMe | C |
| P20 | CH₂CH₃ | OMe | C |
| P21 | CH₃ | NH₂ | A |
| P22 | CH₃ | NHMe | A |
| P23 | CH₃ | NMe₂ | A |
| P24 | CH₃ | SMe | A |
| P25 | CH₃ | OMe | A |

HCV Replicon Activity of 3′-beta-6-Substituted Adenosines

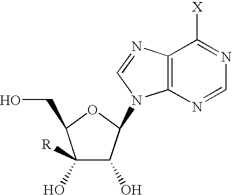

| Compound | 3′-beta-R | 6-X | HCV replicon EC₅₀ |
|---|---|---|---|
| P26 | —C≡CH | NHMe | C |
| P27 | —C≡CH | NMe₂ | C |
| P28 | —C≡CH | SMe | C |
| P29 | CH₃ | NHMe | C |
| P30 | CH₃ | NMe₂ | C |
| P31 | CH₃ | SMe | C |

HCV Replicon Activity of 3′-alpha-6-substituted Adenosines

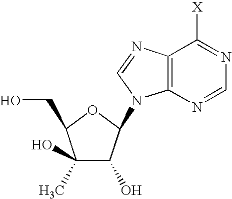

| | | |
|---|---|---|
| Compound | 6-X | HCV Replicon EC₅₀ |

| | | |
|---|---|---|
| P32 | NHMe | C |
| P33 | NMe₂ | C |
| P34 | SMe | C |

HCV Replicon Activity of 4′-alpha-6-substituted Adenosines

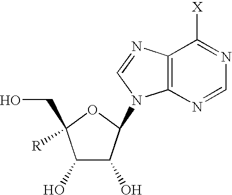

| Compound | R | X | HCV Replicon EC₅₀ |
|---|---|---|---|
| P35 | CH₂CH₃ | NH₂ | C |
| P36 | CH₂CH₃ | NMe₂ | B |

HCV Replicon Activity of 2′-beta-Methyl Cytidine and Uridine
Derivatives

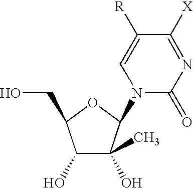

| COMPOUND | R | X | HCV REPLICON EC₅₀ |
|---|---|---|---|
| P37 | H | NH₂ | A |
| P38 | CH₃ | NH₂ | C |
| P39 | H | OH | C |
| P40 | H | NHMe | C |
| P41 | H | NMe₂ | C |
| P42 | CH₃ | OH | C |
| P43 | CH₃ | NHMe | C |
| P44 | CH₃ | NMe₂ | C |

### Pharmacokinetic and Toxicity Data

6-Thiomethyl-9H-(2′-C-methyl-β-D-ribofuranosyl)adenine (P24) shows C₀ of 3452.4 ng/mL, AUC of 1950.6 hr×ng/mL and t_{1/2b} of 0.43 hr for intravenous dosing; and Cₘₐₓ of 1085.0 ng/mL, Tₘₐₓ of 0.75 hours, AUC of 1953.5 hr×ng/mL and t_{1/2β} of 1.19 hours for oral dosing. Therefore, it shows the bioavailability of 100% that increases approximately 4 times comparing to the bioavailability of Ribavirin (27.1%). Compound P24 did not show toxicity in mice at 160 mg/kg dosing with normal body weight, food consumption, and behavior. No tissue and organ abnormalities were observed.

Biological Assays

The following assays were used to measure the inhibition of HCV, influenza, BVDV, HIV, RSV, HRV, HBV, and cytotoxicity as described below. The inventors have discovered that selected compounds, and particularly selected 2′-beta-methyl nucleoside analogs show good antiviral activities.

### HCV Replicon Assay

The replicon cells (Huh-7) contain replicating HCV replicon RNA, which was modified in the structural region (replacing the structural region with a neomycin resistance marker). Survival of the replicon cells under G418 selection relies on the replication of HCV RNA and subsequently expression of neomycin phosphoryltransferase. The ability of modified nucleoside libraries and compounds to suppress HCV RNA replication was determined using the Quantigene Assay Kit from Bayer. The assay measures the reduction of HCV RNA molecules in the treated cells. Replicon cells were incubated at 37° C. for 3 days in the presence of nucleoside libraries and compounds before harvested for detection. An HCV subgenomic replicon cell line was provided by Dr. Bartenschlager. The assay protocol was modified based on literature procedure (V. Lohmann, F. Korner, J. O. Koch, U. Herian, L. Theilmann, R. Bartenschlager, Science, 1999, 285, 110-113).

### Assay for Inhibition of BVDV

Bovine viral diarrhea virus (BVDV) (strain NADL) was provided by Dr. Ruben Donis and propagated in MDBK cells (ATCC). The nucleoside libraries and compounds were tested utilizing the modified protocol (V. B. Vassilev, M. S. Collett, R. O. Donis, J. Viol. 1997, 71, 471-478; S. G. Bagginski, D. C. Pevear, M. Seipel, S. C. C. Sun, C. A. Benetatos, S. K. Chunduru, C. M. Rice, M. S. Collett, Proc. Natl Acad. Sci. U.S.A. 2000, 97, 7981-7986)

### Hepatitis B Virus (HBV) Assay

The in vitro anti-HBV activity of nucleoside libraries and compounds can be tested based on the reported protocol (W. E. Delaney, 4^{th}, R. Edwards, D. Colledge, T. Shaw, J. Torresi, T. G. Miller, H. C. Isom, C. T. Bock, M. P. Manns, C. Trautwein, S. Locarnini, Antimicrob. Agents Chemother., 2001, 45, 1705-1713; W. E. Delaney, 4^{th}, T. G. Miller, H. C. Isom, Antimicrob. Agents Chemother., 1999, 43, 2017-2026; B. E. Korba, J. L. Gerin, Antiviral Res., 1992, 19, 55-70).

### Human Immunodeficiency Virus (HIV) Assay

The in vitro HIV-1 activity of nucleoside libraries and compounds can be tested utilizing the following modified protocol. Freshly isolated human PBMCs from healthy donors are infected with HIV-1 isolates for 3 hours. The cells are then washed three times to remove the viruses. The infected cells are plated into 96-well tissue culture plates and incubated for 7 days in the presence of serially diluted nucleoside analogues (with a medium change at day 4). A standardized HIV-1 p24 Elisa is performed to measure the extent of HIV replication in the presence of the compounds. (C. J. Petropoulos, N. T. Parkin, K. L. Limoli, Y. S. Lie, T. Wrin, W. Huang, H. Tian, D. Smith, G. A. Winslow, D. J. Capon, J. M. Whitcomb, Antimicrob. Agents Chemother., 2000, 44, 920-928; Parkin, N. T., Y. S. Lie, N. Hellmann, M. Markowitz, S. Bonhoeffer, D. D. Ho, C. J. Petropoulos, J. Infect. Disease, 1999, 180, 865-870).

### Human Rhinovirus (HRV) Assay

The in vitro activity of nucleoside libraries and compounds against HRV can be tested based on the reported protocol (W.-M. Lee, W. Wang, R. Rueckert, Virus Genes, 1994, 9, 177-181; B. Sherry, R. Rueckert, J. Virol. 1985, 53, 137-143).

### Respiratory Syncytial Virus (RSV) Assay

The RSV activity of nucleoside libraries and compounds can be tested based on the reported protocol. Respiratory syncytial virus (strain A-2) is purchased from ATCC and virus stock is obtained by propagating the virus in Hep-2 cells. (P. R. Wyde, L. R. Meyerson, B. E. Gilbert, Drug Dev. Res. 1993, 28, 467-472).

### Yellow Fever Virus (YFV) Assay

Yellow fever virus (vaccine strain 17-D) is purchased from ATCC (VR-1 268) and the virus stock is obtained by infecting SW-13 cells from ATCC. The YFV activity of nucleoside libraries and compounds can be tested utilizing the reported protocol (J. J. Schlesinger, S. Chapman, A. Nestorowicz, C. M. Rice, T. E. Ginocchio, T. J. Chambers, J. Gen. Virol. 1996, 77, 1277-1285).

### Influenza Virus Assay

Influenza virus (type A, A/PR/8/34) is produced by infecting pathogen-free, fertilized chicken eggs. The antiviral assay can be performed on Madin Darby canine kidney (MDCK) cells from ATCC based on the reported protocol (E. H. Nasser, A. K. Judd, A. Sanchez, D. Anastasion, D. J. Bucher, J. Virol. 1996, 70, 8639-8644).

### Cytotoxicity Assay

The cytotoxicity of nucleoside libraries and compounds was measured by the MTS cell-based assay from Promega (CellTiter 96 Aqueous One Solution Cell Proliferation Assay).

Thus, specific embodiments and applications of sugar modified nucleosides as viral RNA replication inhibitors have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A compound according to Formula 1 or Formula 2: 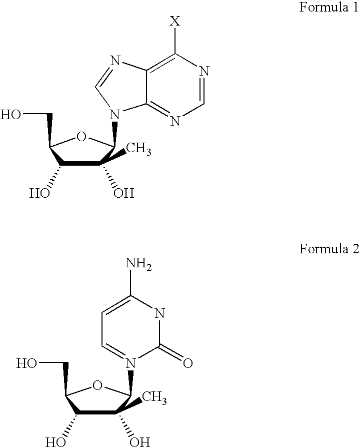
wherein X is selected from the group consisting of NH₂, NHCH₃, N(CH₃)₂, OCH₃, and SCH₃.

2. The compound of claim 1 further comprising a moiety covalently coupled to at least one of the C2′-atom, C3′-atom, and C5′-atom, and wherein at least part of the moiety is preferentially cleaved from the compound in a target cell or target organ.

3. The compound of claim 2 wherein the moiety comprises a cyclic phosphate, a cyclic phosphonate or a cyclic phosphoamidate.

4. The compound of claim 2 wherein the moiety has a structure according to Formula M1 or Formula M2 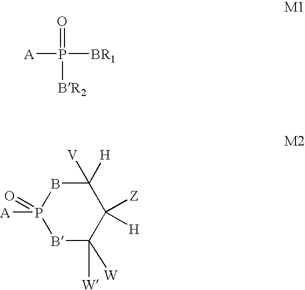
wherein A in M1 or M2 is O or CH₂ and replaces the 5′-OH group of the compound of Formula 1 or Formula 2;
B and B′ are independently O or NH, and where B is NH then R₁ or R2 is an amino acid that forms a peptide bond with the N atom of the NH; and
V, W, and W′ are independently hydrogen, alkyl, alkenyl, alkynyl, aryl, alkaryl, each of which is optionally substituted, and Z is hydrogen, CHWOH, CHWOCOW′, SW, or CH₂aryl.

5. A pharmaceutical composition comprising a compound of Formula 1 or Formula 2: 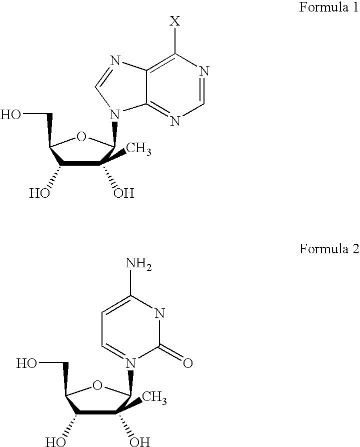
wherein X is selected from the group consisting of NH₂, NHCH₃, N(CH₃)₂, OCH₃, and SCH₃; and
wherein the compound is present in the composition at a concentration effective to inhibit viral RNA replication.

6. The composition of claim 5 wherein the compound further comprises a moiety covalently coupled to at least one of the C2′-atom, C3′-atom, and C5′-atom, and wherein at least part of the moiety is preferentially cleaved from the compound in a target cell or target organ.

7. The composition of claim 6 wherein the moiety comprises a cyclic phosphate, a cyclic phosphonate or a cyclic phosphoamidate.

8. The composition of claim 6 wherein the moiety has a structure according to Formula M1 or Formula M2 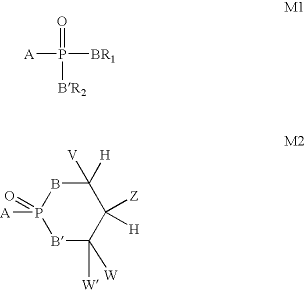
wherein A in M1 or M2 is O or CH₂ and replaces the 5′-OH group of the compound of Formula 1 or Formula 2;
B and B′ are independently O or NH, and where B is NH then R₁ or R2 is an amino acid that forms a peptide bond with the N atom of the NH; and
V, W, and W′ are independently hydrogen, alkyl, alkenyl, alkynyl, aryl, alkaryl, each of which is optionally substituted, and Z is hydrogen, CHWOH, CHWOCOW′, SW, or CH₂aryl.

9. The composition of claim 5 wherein X comprises a nitrogen atom.

10. The composition of claim 5 wherein X is OCH₃ or SCH₃.

11. The composition of claim 5 wherein viral RNA replication is that of HCV.

12. The composition of claim 11 wherein hepatitis C virus replication is mediated by an RNA-dependent RNA polymerase.

13. A method of treating a viral infection in a mammal comprising:
presenting a compound according to Formula 1 or Formula 2 to a cell of the mammal infected with a virus at a concentration effective to reduce viral propagation; 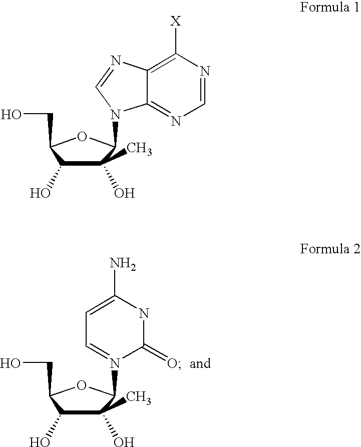
wherein X is selected from the group consisting of NH₂, NHCH₃, N(CH₃)₂, OCH₃, and SCH₃.

14. The method of claim 13 wherein the viral infection comprises an organ inflammation.

15. The method of claim 13 wherein the cell is a hepatocyte.

16. The method of claim 13 wherein the virus is a member of the Flaviviridae.

17. The method of claim 13 wherein the virus is a hepatitis C virus.

18. The method of claim 13 wherein the step of presenting comprises intracellular presentation.

19. The method of claim 13 further comprising administering the compound as a prodrug to the mammal, wherein the prodrug is converted to the compound in the mammal.

20. The method of claim 19 wherein the prodrug is preferentially converted to the compound in the liver.

21. The method of claim 19 wherein the prodrug comprises an ester bond that is cleaved to yield the compound.

22. The method of claim 21 wherein the prodrug comprises a cyclic phosphate, a cyclic phosphonate or a cyclic phosphoamidate.

23. The method of claim 21 wherein the prodrug comprises a moiety having a structure according to Formula M1 or Formula M2 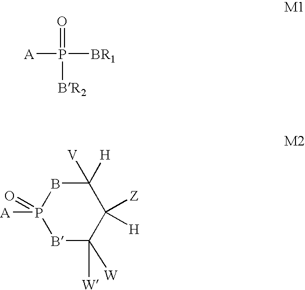
wherein A in M1 or M2 is O or CH₂ and replaces the 5′-OH group of the compound of Formula 1 or Formula 2;
B and B′ are independently O or NH, and where B is NH then R₁ or R2 is an amino acid that forms a peptide bond with the N atom of the NH; and
V, W, and W′ are independently hydrogen, alkyl, alkenyl, alkynyl, aryl, alkaryl, each of which is optionally substituted, and Z is hydrogen, CHWOH, CHWOCOW′, SW, or CH₂aryl.

24. The method of claim 13 further comprising, administration of a second pharmacological molecule.

25. The method of claim 24 wherein the second pharmacological molecule is selected from the group consisting of ribavirin, interferon-alpha, interferon-gamma, and a molecule that induces expression of a interferon-alpha or interferon-gamma in the mammal.
